(19) United States
(12) Patent Application Publication
(10) Pub. No.: US 2025/0129375 A1
(43) Pub. Date: Apr. 24, 2025

(54) CARBON DIOXIDE-CONSUMING EXPRESSION SYSTEM

(71) Applicant: 350 PPM BIOTECH GMBH, Hamburg (DE)

(72) Inventors: Matthias MACK, Oftersheim (DE); Benjamin KACHEL, Mannheim (DE); Charlotte KASPAR, Dossenheim (DE)

(21) Appl. No.: 18/688,431

(22) PCT Filed: Sep. 4, 2022
(86) PCT No.: PCT/EP2022/074540

§ 371 Date: Mar. 1, 2024

(30) Foreign Application Priority Data
Sep. 4, 2021 (EP) 21194944.1

(51) Int. Cl. C12N 15/74; C07K 14/61; C12N 1/20; C12R 1/01

(52) U.S. Cl. CPC C12N 15/74; C07K 14/61; C12N 1/205; C12R 2001/01

(57) ABSTRACT

The present invention generally relates to the field of heterologous protein production in host cells. In particular, the invention relates to a chemolithoautotrophic bacterium which has been genetically modified to produce one or more heterologous proteins. The invention also relates to a method for heterologous protein expression which makes use of the genetically modified chemolithoautotrophic bacterium of the invention. The invention further relates to the use of the genetically modified chemolithoautotrophic bacterium of the invention for heterologous protein expression. The invention also provides a kit which comprises the genetically modified chemolithoautotrophic bacterium of the invention. Finally, the invention relates to a method of introducing an exogenous nucleic acid molecule into a chemolithoautotrophic bacterium.

### RELATED APPLICATIONS

This application is the U.S. National Stage of International Patent Application No. PCT/EP2022/074540, filed Sep. 4, 2022, which is hereby incorporated by reference in its entirety, and which claims priority to EP patent application Ser. No. 21/194,944.1, filed Sep. 4, 2021.

### SEQUENCE LISTING

This application contains a sequence listing submitted electronically as a Standard ST.26 compliant XML file, with a file name of “P 113462 SEQ LIST_Rev.xml,” a creation date of May 2, 2024, and a file size of 95,106 bytes, the contents of which are incorporated herein.

### FIELD

The present invention generally relates to the field of heterologous protein production in host cells. In particular, the invention relates to a chemolithoautotrophic bacterium which has been genetically modified to produce one or more heterologous proteins. The invention also relates to a method for heterologous protein expression which makes use of the genetically modified chemolithoautotrophic bacterium of the invention. The invention further relates to the use of the genetically modified chemolithoautotrophic bacterium of the invention for heterologous protein expression. The invention also provides a kit which comprises the genetically modified chemolithoautotrophic bacterium of the invention. Finally, the invention relates to a method of introducing an exogenous nucleic acid molecule into a chemolithoautotrophic bacterium.

### BACKGROUND OF THE INVENTION

It is widely recognized that human-induced carbon dioxide (CO₂) emission is the main cause of global warming. The German Federal Environment Agency estimated that industrial processes in Germany alone produce around 45 million tons of CO₂ emissions annually. To prevent the worst effects of global warming, international treaties were negotiated and adopted that sought to limit future emissions of greenhouse gases that contribute to global warming. Most significantly, the long-term temperature goal of the Paris climate agreement is to keep the rise in mean global temperature below 2° C., and preferably below 1.5° C. above pre-industrial levels.

In the Intergovernmental Panel on Climate Change (IPCC) climate report of 2021, it is assumed that it will not be possible to reach the 1.5° C., temperature goal without so-called “negative emissions”, i.e, the active removal of CO₂ from the atmosphere. One simple way of removing CO₂ from the atmosphere resides in afforestation. According to studies, 3.6 billion tons of CO₂ per year could be bound during the growth phase, which corresponds to about 10 percent of the present CO₂ emissions. However, afforestation requires very large areas, which means that areas presently used for agriculture would have to be converted, thereby interfering with the ability to produce food for a growing world population. Storing CO₂ deep underground is another well-known possibility of proving negative emissions. The CO₂ is first extracted from the ambient air by means of chemical processes and subsequently stored in underground geological formation, e.g. at the sea floor. This process is referred to as carbon capture and storage (CCS). Some CCS plants are already operating in Europe. While the CCS technology has great potential, it is presently extremely cost-intensive. The cost for removing a ton of CO₂ by CCS amounts to approximately 500-550 Euros. At present, the high costs are a barrier for using CCS in large scale.

Another idea that was discussed at a scientific level is the fertilization of the oceans with iron compounds. The rationale is to increase the nutrient content in the oceans such that plankton growth can be significantly increased which in turn leads to increased CO₂ binding. However, this idea is considered to involve a serious intervention into the ocean ecosystem which may be associated with severe and unpredictable side effects.

Another potential approach for providing negative emissions is the deployment of biotechnological processes that involve the consumption of CO₂. For example, it is known that certain microorganisms are able to convert CO₂ into organic carbon compounds. For example, it has been reported that autotrophic methanogenic archaea can consume CO₂ by converting it into methane. However, methanogenic archaea appear not to be suited for making products other than methane, another compound which contributes to global warming. Another group of microorganisms that efficiently utilize CO₂ are photoautotrophic bacteria, and in particular cyanobacteria (Kachel & Mack, 2020). However, these organisms grow relatively slowly which is mainly due to the technically difficult input of light energy.

Yet another interesting approach for CO₂ utilization is already in the pilot stage. The ‘Rheticus’ project jointly carried out by Siemens and Evonik aims at the biotechnological production of butanol and hexanol by chemolithotrophic organisms (Hoff et al., 2021). The technology uses electricity from renewable energy for the conversion of CO₂ into CO and H₂. CO₂, CO, and H₂ are fed into a three-stage fermentation process designed by Evonik using Clostridium autoethanogenum and Clostridium kluyveri finally generating a 1:1 mixture of butanol and hexanol. This mixture can either be separated by distillation or be employed as a fuel mixture. It is estimated that within five years, an industrial plant using this technology can produce 10.000 tons of hexanol and butanol per year using 25,000 tons of CO₂ as starting material.

In light of the high amounts of CO₂ that are emitted in the industrial countries, there is a high need for additional biotechnological processes that involve the consumption of CO₂. The biotechnological processes should preferably be feasible on an industrial scale so that as much CO₂ as possible is consumed. The process should be robust and easy to establish. In addition, the process should result in the production of a valuable product that can be used, e.g. in medicine, or as a food or feed.

The present invention addresses this need and provides additional advantages as well. The invention provides an expression host which can serve as a platform for establishing sustainable, CO₂-consuming biotechnological production processes. Importantly, the expression host can be genetically modified to produce one or more heterologous proteins, in particular human proteins. The expression host is a chemolithoautotrophic bacterium which is able to grow in the presence of CO₂ as a carbon source and hydrogen (H₂) as an energy source. Since the final acceptor for the reducing equivalents generated by the respiratory chain can be oxygen (O₂), the expression host of the present invention is classified as an aerobic organism.

### DESCRIPTION OF THE INVENTION

Thus, in a first aspect, the present invention provides a chemolithoautotrophic bacterium of the genus Hydrogenovibrio which has been genetically modified to produce one or more het30erologous proteins. Preferably, the genetically modified chemolithoautotrophic bacterium belongs to the species Hydrogenovibrio marinus. More preferably, the genetically modified bacterium is derived from H. marinus MH-110, deposited at the Japanese Cell Collection (JCM) under deposition number JCM 7688. Bacterial microorganisms of the genus Hydrogenovibrio have high CO₂ binding capacities which render them suitable for use in a 35 method of the present invention. For example, wild-type H. marinus contains three different ribulose 1,5-bisphosphate carboxylase/oxygenases (RubisCOs) which allow the organism to adapt to different CO₂ levels in the environment (Toyoda et al., 2005: Toyoda et al., 2018). While known aerobic hydrogen-oxidizing bacteria are sensitive to high oxygen levels and grow comparably slowly. H. marinus is able to grow rather quickly, up to a pO₂ of 40%. Optimum growth was observed at a pO₂ between 5 and 10%. The complete genome of H. marinus has been sequenced (Arai et al., 2019; Jo et al., 2014).

H. marinus MH-110 was isolated from a seawater sample collected at the coast of Shonan in Japan. At the time of isolation, H. marinus MH-110 was the first known mesophilic, obligate chemolithoautotrophic and aerobic hydrogen-oxidizing bacterium. The optimal observed growth temperature is 37° C. At 5° C. or 45° C. no growth is observed. As a marine bacterium, H. marinus grows optimally in an aqueous growth medium at about 0.5-0.6 M NaCl with a pH preference of around 6.5. In principle, seawater of a suitable salinity can be used to prepare growth media for potential biotechnological production processes employing H. marinus which has the advantage that the risk for microbial contamination is further reduced. Notably, not many contaminating microorganisms are able to grow under chemolithoautotrophic conditions. Moreover, the use of seawater may be advantageous as it can be used in regions where freshwater is scarce.

The genetically modified bacteria of the invention are chemolithoautotrophic which means that they obtain the energy required for their growth from the oxidation of inorganic compounds such as H₂. At the same time, they are autotrophic which means that they are able to produce complex organic compounds using carbon from simple substances. In the case of bacteria from the genus Hydrogenovibrio, H₂ is used as an energy source and CO₂ as a carbon source. The particular advantage of Hydrogenovibrio is that it binds high amounts of CO₂ which means that using the organism in an industrial scale provides for the possibility of removing CO₂ from the ambient air, thereby rendering the process climate-friendly.

The present invention describes means and methods that can be used to generate genetically modified Hydrogenovibrio strains which produce one or more heterologous proteins. As used herein, a heterologous protein is a protein which is not encoded by a gene of the non-modified organism, i.e, the naturally occurring Hydrogenovibrio strain, and therefore not produced by the non-modified organism. The overproduction and extraction of a heterologous protein in cells of the genus Hydrogenovibrio has not been described so far. Hence, the present invention for the first time describes the generation of so-called production strains that can be used in large-scale biotechnological production processes. These strains allow the fully sustainable production of heterologous proteins from CO₂ and hydrogen gas. Preferably, the chemolithoautotrophic bacterium of the present invention has been genetically modified to produce a mammalian, more preferably a human protein. The protein preferably is a therapeutically active protein, i.e. a protein that can be used in human or veterinary medicine.

The type of therapeutically active protein is not particularly limited and can include cytokines, hormones, growth factors, and the like. In one embodiment, the host cells and methods of the invention are useful for the recombinant expression of an interferon protein. The type of interferon is not particularly limited. Generally, the interferon proteins selected for expression in the host cells of the invention can be of human or non-human origin. The interferon protein can belong to any interferon subclass that is known in the art. In particular, the interferon protein to be expressed in the host cell of the invention can be an alpha-interferon (IFN-α), a beta-interferon (IFN-α) or a gamma-interferon (IFN-γ). In a particularly preferred embodiment. the interferon protein to be expressed is human IFN-α, IFN-β or IFN-γ or a biologically active fragment thereof.

In a preferred embodiment, the mammalian protein is human somatotropin (hGH) or a biologically active fragment thereof. As shown herein, it was possible to create a H. marinus strain that produces human somatotropin, a protein that is also referred to in the literature as human growth hormone (hGH). Somatotropin is a protein that is produced by the pituitary gland in the brain and stimulates the liver to produce somatomedins, i.e. hormones that are very similar in structure to insulin and stimulate the growth of bones. Children and adolescents normally produce large quantities of somatotropin. hGH is normally produced as a precursor of 217 amino acids. The major isoform of the hGH is a protein of 191 amino acids and a molecular weight of 22,124 daltons. The structure includes four helices necessary for functional interaction with its cognate growth hormone receptor. Recombinant hGH (rhGH) was shown to be 25 biologically active when produced in prokaryotic systems e.g. in Escherichia coli (Olson et al., 1981). This indicates that posttranslational modifications which occur in e.g. mammalian cells are not necessary to generate an active pharmaceutical ingredient. rhGH produced in E. coli was approved by the FDA in 1985. As of 2005, different rhGH products were available in the market from manufacturers like Genentech, Eli Lilly, Pfizer, Merck Serono, and Sandoz.

In a preferred embodiment the chemolithoautotrophic bacterium of the present invention has been modified to produce an hGH protein that comprises or consists of
(a) the amino acid sequence of SEQ ID NO:1, or
(b) an amino acid sequence having at least 80% sequence identity to the amino acid sequences of SEQ ID NO: 1.

Preferably, the hGH protein produced by the chemolithoautotrophic bacterium of the present invention is the protein depicted in SEQ ID NO: 1. Alternatively, the hGH protein produced by the chemolithoautotrophic bacterium can be a variant of the protein depicted in SEQ ID NO: 1 shares a high degree of sequence identity of at least 80%. More preferably, the sequence identity is 91%. 92%. 93%, 94%. 95%. 96%. 97%. 98%, or 99%. Preferably, the sequence identity is determined over a length of at least 50 amino acids, more preferably at least 75 amino acids, at least 100 amino acids, at least 125 amino acids, at least 150 amino acids, at least 175 amino acids, or at least 200 amino acids. More preferably, the sequence identity is determined over a full length of the precursor protein, i.e. 217 amino acids.

In one embodiment, the hGH variant shares a sequence identity with the amino acid sequence of SEQ ID NO:1 of at least 95% over a length of at least 50 amino acids, preferably at least 75 amino acids, more preferably at least 100 amino acids, even more preferably at least 125 amino acids, even more preferably at least 150 amino acids, even more preferably at least 175 amino acids, and even more even more preferably at least 200 amino acids. It is particularly preferred that the sequence identity is at least 95% over the full length of the protein. In one embodiment, the hGH variant shares a sequence identity with the amino acid sequence of SEQ ID NO:1 of at least 96% over a length of at least 50 amino acids, preferably at least 75 amino acids, more preferably at least 100 amino acids, even more preferably at least 125 amino acids, even more preferably at least 150 amino acids, even more preferably at least 175 amino acids, and even more preferably at least 200 amino acids. It is particularly preferred 25 that the sequence identity is at least 96% over the full length of the protein. In one embodiment. the hGH variant shares a sequence identity with the amino acid sequence of SEQ ID NO:1 of at least 97% over a length of at least 50 amino acids, preferably at least 75 amino acids, more preferably at least 100 amino acids, even more preferably at least 125 amino acids. even more preferably at least 150 amino acids, even more preferably at least 175 amino 30 acids, and even more preferably at least 200 amino acids. It is particularly preferred that the sequence identity is at least 97% over the full length of the protein. In one embodiment, the hGH variant shares a sequence identity with the amino acid sequence of SEQ ID NO:1 of at least 98% over a length of at least 50 amino acids, preferably at least 75 amino acids, more preferably at least 100 amino acids, even more preferably at least 125 amino acids, even more 35 preferably at least 150 amino acids, even more preferably at least 175 amino acids, and even more preferably at least 200 amino acids. It is particularly preferred that the sequence identity is at least 98% over the full length of the protein. In one embodiment, the hGH variant shares a sequence identity with the amino acid sequence of SEQ ID NO:1 of at least 99% over a length of at least 50 amino acids, preferably at least 75 amino acids, more preferably at least 100 amino acids, even more preferably at least 125 amino acids, even more preferably at least 150 amino acids, even more preferably at least 175 amino acids, and even more preferably at least 200 amino acids. It is particularly preferred that the sequence identity is at least 99% over the full length of the protein.

In order to determine the sequence identity between two amino acid sequences, these sequences are usually aligned for optimal comparison. For example, gaps can be introduced in the sequence of a first amino acid sequence for optimal alignment with a second amino acid sequence. The amino acids at corresponding positions are then compared. If identical amino acids occur in corresponding positions in the first and second amino acid sequence, the sequences are identical at that position. A percentage sequence identity between two amino acid sequences means that, when aligned, the recited percentage of amino acids are identical in comparing both sequences. A percentage sequence identity can be determined by using software programs that are widely known in the art, for example the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used.

Variants of the protein depicted in SEQ ID NO:1 also include proteins which differ from the original hGH by one or more (e.g. 2. 3, 4, 5, 10, or 15) additional amino acids. These additional amino acids may be present within the amino acid sequence of the original hGH protein (i.e, as an insertion), or they may be added to one or both termini of the protein. Basically, insertions can take place at any position provided that the addition of amino acids does not impair the capability of the protein to exert at least part of the biological activity that is exerted by the naturally occurring hGH protein. Similarly, variants also comprise those proteins in which, compared to the original protein, one or more amino acids are lacking. Such deletions may affect any amino acid position provided that they do not impair the capability of the variant to exert at least part of the biological activity that is exerted by the naturally occurring hGH protein.

Variants of the hGH protein also encompass proteins with structural modifications relative to the naturally occurring protein, such as modified amino acids. According to the invention, modified amino acids may comprise phosphorylation, glycosylation, acetylation, acylation, branching, ADP ribosylation, crosslinking, disulfide bridge formation, formylation, hydroxylation, carboxylation, methylation, demethylation, amidation, cyclization and/or covalent or non-covalent bonding to phosphatidylinositol, flavine derivatives, lipoteichonic acids, fatty acids or lipids. Such modifications have been extensively described in the literature, e.g., in Proteins: Structure and Molecular Properties, T. Creighton, 2^{nd} edition, W. H. Freeman and Company, New York (1993).

In a preferred embodiment, the chemolithoautotrophic bacterium of the invention comprises a plasmid. As used herein, a plasmid refers to an extrachromosomal circular DNA capable of autonomous replication in a cell. The plasmid can be an integrating or non-integrating plasmid. However, the use of a plasmid which does not integrate into the genome of the host cell is preferred. In a preferred embodiment the plasmid is an expression vector which means that it carries a nucleotide sequence that encodes the heterologous protein to be produced by the host cell. Expression vectors are typically DNA constructs that are optimized for protein expression and comprise an origin of replication that allows the vector to replicate independently of the host cell. Expression vectors also include regulatory control elements that are functionally linked to the nucleotide sequences to be expressed and control their transcription and/or translation. Suitable control elements include constitutive or controllable prokaryotic promoters, a transcription termination sequence and a ribosome binding site. Expression vectors may also include one or more antibiotic resistance genes which allow transformed cells to be selected by use of media that contain antibiotics.

The plasmid or expression vector comprised by the chemolithoautotrophic bacterium of the invention preferably comprises a synthetic promoter, more preferably a promoter selected from the group of J23119 (SEQ ID NO:8), J23111 (SEQ ID NO:9) and J23114 (SEQ ID NO:10). The plasmid or expression vector comprised by the chemolithoautotrophic bacterium of the invention preferably also comprises a synthetic ribosomal binding site, more preferably a ribosomal binding site selected from the group of BBa_B0034 (SEQ ID NO:11), BBa_B0064 (SEQ ID NO:12), BBa_B0031 (SEQ ID NO:13), and ribDG (SEQ ID NO:14). A plasmid or expression vector comprising the above-recited components preferably is a nonintegrative plasmid.

The expression vector comprised by the chemolithoautotrophic bacterium of the invention further comprises a gene that codes for the heterologous protein. The size of the heterologous protein encoded by the gene is not particularly limited and preferably ranges from 50 to 1200 amino acids, more preferably from 75 to 1000 amino acids, from 100 to 900 amino acids, from 150 to 800 amino acids, from 175 to 750 amino acids, or from 200 to 600 amino acids. The heterologous protein can encode a therapeutically or enzymatically active or a fragment of such a protein, provide that the fragment is still therapeutically or enzymatically active.

The plasmid or expression vector can be cloned in accordance with routine methods known in the art which are described, e.g, in Maniatis et al. 1982, Molecular Cloning, A laboratory Manual, Cold Spring Harbor Laboratory. The plasmid or expression vector is preferably introduced into the chemolithoautotrophic bacterium of the invention by parental mating with a suitable donor strain. For this purpose, the plasmid or expression vector is first introduced into a E. coli stain, such as E. coli WM3064 by routine methods, such as electroporation or calcium phosphate-or rubidium chloride-mediated transformation. In a subsequent step, the plasmid or expression vector is transferred from the donor strain WM3064 to the Hydrogenovibrio strain by spreading and incubating aliquots from cultures of both strains on a nitrocellulose filter, as described below in Example 3.

In a second aspect, the invention provides a method for the production of one or more heterologous proteins in a host cell, said method comprising the following steps
(a) providing a chemolithoautotrophic bacterium as described hereinabove:
(b) culturing the host cell under conditions that allow the production of the heterologous protein:
(c) obtaining the heterologous protein.

The method of the invention is directed to the production of one or more heterologous proteins in a host cell. Preferably, the method seeks to produce a single heterologous protein. The method makes use of genetically modified chemolithoautotrophic Hydrogenovibrio bacterium described above. Preferably, the genetically modified chemolithoautotrophic bacterium belongs to the species Hydrogenovibrio marinus. More preferably, the genetically modified bacterium is derived from H. marinus MH-110, deposited at the Japanese Cell Collection (JCM) under deposition number JCM 7688.

As used herein, a bacterium is considered to belong to the species H. marinus if the 16S rRNA comprises the
(a) the nucleotide sequence of SEQ ID NO:6, or
(b) a nucleotide sequence having at least 99% or more sequence identity to the nucleotide sequences of SEQ ID NO:6.

Preferably, the nucleotide sequence has at least 99% or more sequence identity to the nucleotide sequences of SEQ ID NO:6 over a length of 400 nucleotides, 500 nucleotides, 600 nucleotides, 700 nucleotides, 800 nucleotides, 900 nucleotides, or 1000 nucleotides of the sequence of SEQ ID NO:6. More preferably, the nucleotide sequence has at least 99% or more sequence identity to the nucleotide sequences of SEQ ID NO:6 over the full length of the 1402 nucleotides. More preferably, the nucleotide sequence has at least 99.5% or more sequence identity to the nucleotide sequences of SEQ ID NO:6 over a length of 400 nucleotides, 500 nucleotides, 600 nucleotides, 700 nucleotides, 800 nucleotides, 900 nucleotides, or 1000 nucleotides of the sequence of SEQ ID NO:6. Even more preferably, the nucleotide sequence has at least 99.5% or more sequence identity to the nucleotide sequences of SEQ ID NO:6 over the full length of the 1402 nucleotides. More preferably, the nucleotide sequence has at least 99.8% or more sequence identity to the nucleotide sequences of SEQ ID NO:6 over a length of 400 nucleotides, 500 nucleotides, 600 nucleotides, 700 nucleotides, 800 nucleotides, 900 nucleotides, or 1000 nucleotides of the sequence of SEQ ID NO:6. Even more preferably. the nucleotide sequence has at least 99.8% or more sequence identity to the nucleotide sequences of SEQ ID NO:6 over the full length of the 1402 nucleotides.

The Hydrogenovibrio bacterium of the invention is cultured under conditions that allow the production of the heterologous protein. Such conditions include conditions of temperature, pH and salt that allow for the growth and propagation of the cells. For Hydrogenovibrio marinus, an appropriate growth temperature will be in the range of 30-39° C., whereas a growth temperature of 36-37° C., is particularly preferred. The media used for cell culturing will include a certain amount of salt. Preferably, the media used for culturing contain 0.2-1.0 M NaCl, and more preferably 0.5-0.6 M NaCl. A pH in the range of 6.0-8.0 is preferred, more preferably 7.0-8.0, such as 6.5.

Cell culturing is performed in a gaseous atmosphere containing H₂, CO₂ and oxygen (O₂). A wide variety of different ratios of the three gases can be used for generating the atmosphere used for culturing. Preferably, atmosphere compositions are avoided that contain 4% or more O₂ and, at the same time, 4% or more H₂, since such atmosphere compositions are explosive which complicates the culturing of the cells. It is therefore preferred that either O₂ or H₂ or both are used in an amount of less than 4% when producing the atmosphere compositions for cell culturing.

In one embodiment, H₂ is present in the atmosphere composition for cell culturing in an amount of 80-89%, more preferably 85-89%, and even more preferably 87-89%. In such embodiment, the CO₂ is present in the atmosphere composition in an amount of 2-10%, more preferably 5-10%, and even more preferably 8-10%. In such embodiment, the O₂ is present in the atmosphere composition in an amount of 0.5-3%, more preferably 1-3%, and even more preferably 2-3%. The atmosphere composition may also include N₂ in an amount of up to 2%. Preferred atmosphere compositions include H₂:CO₂:O₂:N₂=89:10:1:0, 88:10:2:0, and 87:10:3:0. Alternative preferred atmosphere compositions include H₂:CO₂:O₂:N₂=82:15:3:0. 83:15:2:0, and 84:15:1:0.

In another embodiment, H₂ is present in the atmosphere composition for cell culturing in an amount of 70-79%, more preferably 75-79%, and even more preferably 77-79%. In such embodiment, the CO₂ is present in the atmosphere composition in an amount of 2-10%, more preferably 5-10%, and even more preferably 8-10%. In such embodiment, the O₂ is present in the atmosphere composition in an amount of 2-10%, more preferably 5-10%, and even more preferably 8-10%. The atmosphere composition may also include N₂ in an amount of up to 5%. Preferred atmosphere compositions include H₂:CO₂:O₂:N₂=77:10:10:3, 78:10:10:2, and 79:10:10:1.

In yet another embodiment, H₂ is present in the atmosphere composition for cell culturing in an amount of 0.5-3%, more preferably 1-3%, and even more preferably 2-3%. In such embodiment, the CO₂ is present in the atmosphere composition in an amount of 2-10%, more preferably 5-10%, and even more preferably 8-10%. In such embodiment, the O₂ is present in the atmosphere composition in an amount of 2-10%, more preferably 5-10%, and even more preferably 8-10%. The atmosphere composition may also include N₂ in an amount of up to 80%. Preferred atmosphere compositions include H₂:CO₂:O₂:N₂=3:9:8:80, 3:8:9:80, 3:9:9:79, and 2:10:10:78.

Conveniently, it is also possible to culture the Hydrogenovibrio strains of the invention in a gaseous atmosphere that comprises ambient air (which includes about 78% N₂, about 21% O₂ and some other gases) that has been supplemented with 8-10% CO₂ and 1-3% H₂. Preferably, the gaseous atmosphere comprises ambient air that has been supplemented with 10% CO₂ and 3% H₂ or ambient air that has been supplemented with 10% CO₂ and 2% H₂.

The Hydrogenovibrio strain of the invention is cultured until a suitable OD has been reached, preferably an OD₅₄₀ of 0.3-1.8, more preferably an OD₅₄₀ of 0.5-1.6, and even preferably an OD₅₄₀ of 0.7-1.0. Once a sufficiently high OD has been reached, the culturing is stopped and the protein is harvested.

In step (c) of the method, the heterologous protein is obtained from the host cell. Typically. the protein is isolated from the cytosol of the host cells. Preferably, step (c) of the method comprises the disruption of the cells, e.g., by a French press or bead mill, by repeating cycles of freezing and thawing, or by sonication. Cellular debris can be easily removed from the protein fraction by centrifugation. The heterologous protein can then be purified from other protein and non-protein components by standard chromatography methods, including, for example, size exclusion chromatography and/or ion exchange chromatography. Other methods which are typically applied in protein purification, such as ammonium sulfate precipitation and filtration and/or dialysis may also be used. The heterologous protein expressed by the host cell of the invention may also be purified by affinity purification using, e.g., monoclonal or polyclonal antibodies.

In a preferred embodiment, step (c) of the method of the invention comprises the purification of the heterologous protein. To simplify purification, the heterologous protein produced according to the method of the invention may be expressed as a fusion protein which comprises an affinity tag that facilitates binding of the fusion protein via a compound exhibiting binding affinity to the tag. For example, the affinity tag may be a poly-histidine tag comprising 6-12 histidine residues which specifically interacts with a nickel ion chelate matrix. Alternatively, the tag may be glutathione-S-transferase allowing the purification on a glutathione matrix. Further affinity tags are well-known in the art. Non-limiting examples for pairs of affinity tag and affinity ligand include maltose-binding-protein (MBP) and maltose: avidin and biotin: Streptag and streptavidin: myc epitope and antibody. Where the affinity tag is a peptide or polypeptide, such tag may conveniently be expressed together with the heterologous protein as a single expression product. Where the affinity tag is not of protein origin, it may be attached by chemical coupling reactions to the heterologous protein. For example, biotin may be chemically coupled to the heterologous protein.

In the event that the genetically modified Hydrogenovibrio bacterium of the invention is used for the production of a therapeutically or enzymatically active protein, it is preferred that at least 50% of the heterologous protein produced according to the method of the invention is therapeutically or enzymatically active protein. More preferably, at least 60%, at least 70%, at least 80%, at least 90%, or at least 95% of the heterologous protein obtained from the method of the invention is therapeutically or enzymatically active.

In a third aspect, the invention relates to the use of the chemolithoautotrophic bacterium described above as a host cell for the production of a heterologous protein.

In a fourth aspect, the invention relates to a kit for carrying out a method. In particular, the invention relates to a kit for the production of a heterologous protein comprising the chemolithoautotrophic bacterium described above. The kit comprises the chemolithoautotrophic bacterium described hereinabove, i.e, a chemolithoautotrophic Hydrogenovibrio bacterium which has been genetically modified to produce one or more heterologous proteins. The kit further comprises media and reagents that are suitable for culturing the Hydrogenovibrio bacterium and initiating gene expression.

It was also surprisingly found in the course of the present invention that marine chemolithoautotrophic bacteria can be transformed by electroporation, if certain prerequisites are met. This finding is unexpected as marine bacteria require salt in the surrounding medium which is normally not compatible with electroporation. Most importantly, it appears that the cells tolerate the treatment under high-voltage conditions if ectoine is added to the medium used for electroporation. This is an important insight, since alternative transformation methods are far more laborious. Therefore, in a fifth aspect, the invention relates to a method of introducing an exogenous nucleic acid molecule into a chemolithoautotrophic bacterium, said method comprising:
a) preparing a suspension of bacterial cells of the genus Hydrogenovibrio in a fluid that comprises ectoine and an exogenous nucleic acid molecule, and
b) electroporating the bacterial cells to introduce the exogenous nucleic acid molecule in the cells.

Preferably, the cells to be transformed belong to the genus Hydrogenovibrio, and more preferably to the species of H. marinus. The cells to be transformed are normally first cultured under suitable conditions. Suitable media and conditions that allow the growth of Hydrogenovibrio cells have been described in detail above. The cells are preferably cultured for 8-16 hours. Subsequently, aliquots of the culture are withdrawn for electroporation. It is preferred that the cells are diluted to an OD₅₄₀=below 0.1, and more preferably to an OD₅₄₀ in the range of 0.04 to 0.06. The dilution can be effected by the addition of fresh medium. The cells are then usually harvested by centrifugation and concentrated to a high OD₅₄₀ value, preferably a value in the range of 3-5, and more preferably 5. After centrifugation, the pelleted cells are then suspended in a fluid that comprises ectoine and an exogenous nucleic acid molecule.

The fluid preferably is a solution of ectoine in water, e.g, a solution comprising ectoine in a concentration of between 0.25 and 2.5 mol/L, and preferably between 0.5 and 2.0 mol/L. In one embodiment, the ectoine is present in the fluid in a concentration of 0.25 mol/L or more, 0.50 mol/L or more, 0.75 mol/L or more, 1.00 mol/L or more, 1.25 mol/L or more, 1.50) mol/L or more, 1.75 mol/L or more, 2.00 mol/L or more, 2.25 mol/L or more, or 2.50 mol/L. In a particular preferred embodiment, the ectoine is present in the fluid in a concentration between 0.50 mol/L and 1.00 mol/L. The centrifugation and resuspension steps may be repeated several times, e.g. 2-5 times.

Shortly before electroporation, the nucleic acid to be introduced into the cells is added to the suspension. The nucleic acid is an exogenous nucleic acid which means that it does not naturally occur in the bacterial cell to be transformed. Preferably, the exogenous nucleic acid is a recombinant nucleic acid, i.e, a nucleic acid that has been genetically modified by human intervention. The exogenous nucleic acid can be DNA or RNA, and preferably will be DNA. The nucleic acid may be a linear nucleic acid molecule, such as a linear DNA molecule, or a plasmid or an expression vector, as defined elsewhere herein. The nucleic acid can be added in a suitable amount for electroporation, e.g, in an amount of 0.5-50 ng per μl cell suspension, such as 1-3 ng per μl cell suspension. Electroporation can be conducted under known conditions using standard device, such as a Gene Pulser II (Biorad).

Immediately after electroporation, the cells are supplemented with fresh culture medium. For example, if the bacteria to be transformed belong to the genus Hydrogenovibrio, the medium described above can be used in this step. It has been found that the viability of the cells after electroporation can be significantly increased if the medium that is added has been previously incubated under an atmosphere that comprises or essentially consists of H₂ and CO₂. The atmosphere preferably comprises at least 70% (v/v), at least 75% (v/v), at least 80% (v/v), at least 85% (v/v), at least 90% (v/v), or at least 95% (v/v) H₂. It is further preferred that the atmosphere in which the culture medium is incubated comprises at least 0.5% (v/v), at least 0.75% (v/v), at least 1.0% (v/v), at least 1.5% (v/v), at least 2.0% (v/v), at least 2.5% (v/v), or at least 3.0% (v/v) CO₂. In a particular preferred embodiment, the atmosphere essentially consists of H₂ and CO₂, which means that it does not comprise more than 0.5% (v/v) of any other gas. For example, the atmosphere may consist of 96.7% H₂ and 3.3% CO₂. The medium is incubated under the H₂- and CO₂-containing atmosphere for at least 2 hours, and preferably for at least 4 hours, for at least 6 hours, for at least 8 hours, for at least 10 hours, or at least 12 hours before adding it to the bacterial cells to be transformed.

Accordingly, in a preferred embodiment, the method of the invention further comprises:
c) after electroporation, culturing the bacterial cells in a culture medium that was kept in an atmosphere that essentially consists of H₂ and CO₂ for at least 2 hours before addition to the cells.

After addition of the culture medium, the cells may be further cultured under suitable conditions. Suitable conditions for culturing Hydrogenovibrio have been described above and include the culturing of the cells in a gaseous atmosphere containing H₂, CO₂ and O₂ at a variety of different ratios, as described above. For example, the cultures can be incubated for 6 to 7 days in an atmosphere of H₂:CO₂:O₂=87:10:3 at 37° C. The gas atmosphere is preferably exchanged every 1-3 days.

After reaching a suitable optical density, the cells obtained from electroporation can be stored until further use or until further analysis. For example, the cells may be analyzed by PCR or they may be selected by using a selection marker that is encoded by the nucleic acid that was introduced into the cells.

### BRIEF DESCRIPTION OF THE FIGURES

FIG. 1 shows the production of sf-GFP in Hydrogenovibrio marinus. (A) Phase contrast microscopy confirms that cells of a recombinant H. marinus strain containing the expression plasmid pHM38 show the same characteristic shape as the wild-type. (B). Fluorescence of sfGFP was excited at a wavelength of 485 nm and emission was captured at 525 nm. (C) SDSPAGE analysis followed by Coomassie Brilliant Blue R-250 staining of the soluble fraction of recombinant H. marinus cell lysates reveals a strong protein band at about 26 kDa in case of the H. marinus strain expressing J23119_GFP (pHM38) corresponding to the expected size of sf-GFP. Cell-free extracts of recombinant E. coli WM3064 carrying either the empty expression vector or pHM38, respectively, contain a similar band with a similar strength. Cellular lysates containing 16 ug of total protein were loaded onto the gel. A bovine serum albumin (BSA) standard was used as a control and in order to estimate the amount of sf-GFP protein in the cellular lysate of the recombinant H. marinus strains (M: PageRuler pre-stained protein standard).

FIG. 2 shows the result of measuring sf-GFP fluorescence in recombinant H. marinus strains expressing the gene encoding sf-GFP under the control of different promoter-RBS variants. Error bars indicate one standard deviation from average of n=4 technical replicates.

FIG. 3 shows the detection of rhGH by Western Blot in cell-free extracts of E. coli and H. marinus. Protein extracts from the insoluble (P) and soluble (L) fractions of the cultures producing either His-tagged rhGH or another His-tagged protein from a moderate promoter (mP) were probed via a HRP-conjugated 6xHis antibody and detected with 3,3′diaminobenzidine (DAB) substrate solution.

FIG. 4 shows the result from measuring growth (left) and GFP fluorescence (right) of H. marinus cultures transformed with RSF1010-based replicative plasmids (pHM38, pHM39) containing GFP expression cassettes. The experiment was repeated on two different days (Exp. 1 and Exp. 2), and therefore the data represent biological replicates. H. marinus WT was transformed with either pHM38-containing kanamycin resistance cassette and GFP under the control of a constitutive strongly active promoter or with pHM39-containing kanamycin resistance cassette and GFP under the control of a constitutive moderately active promoter. As a control (Ctrl.), H. marinus was mixed with plasmid DNA, but was not subjected to electroporation. Cultures were otherwise treated similar to the transformation cultures. Cultures were grown for 6 or 7 days in selective medium containing 15 μg/mL kanamycin.

FIG. 5 shows Growth (left) and GFP fluorescence (right) of H. marinus cultures transformed with an integration cassette targeting the bla locus and containing a GFP expression cassette. The experiment was repeated on two different days (Exp. 1 and Exp. 2), therefore data represent two biological replicates. H. marinus WT was transformed with an integration cassette consisting of a kanamycin resistance cassette (KanR) and GFP under the control of a strong constitutive promoter (J23119_GFP) flanked by two homologous sequences that target integration into the bla locus. Cultures were grown for 6 or 8 days in selective medium containing kanamycin.

FIG. 6 shows the results of colony PCR (cPCR) on genomic DNA of recombinant H. marinus strains containing expression cassettes integrated into the genomic bla region. PCR was conducted with primers DNA_180 (5′-ATACTTGATTCCCGCACACC-3′: SEQ ID NO:31) and DNA_181 (5′-GTCAGTTCAACGAGCAAAGC-3′: SEQ ID NO:32) that bind within regions of the H. marinus genome flanking the bla integration site, but not binding within the integration cassette. A successful integration results in a PCR fragment of about 3.9 kb. Without integration the PCR fragment is about 2.5 kb. Lanes 1-4 show 4 of the 6 strains generated in the transformation described in Example 8. Lane 5) shows a H. marinus strain transformed with the replicative plasmid pHM42 which does not integrate into the genome. Lane 6) shows the H. marinus WT strain. Lanes M) contain GeneRuler™ 1 kb DNA Ladder (Thermo Scientific).

### EXAMPLES

The present invention is further described in more detail by the following examples which are only provided for illustrating the invention and which are not to be construed as limiting the scope of the invention. The following material and methods were used in the Examples.

### Example 1: Culturing of H. marinus

H. marinus strain MH-110 is obtainable from the Japanese Cell Collection (JCM) under deposition number JCM 7688. The strain was cultured in a Basal Medium (Nishihara et al., 1991), with a final composition of 2 g/L K₂HPO₄, 1 g/L KH₂PO₄, 5 g/L (NH₄)₂SO₄, 29.3 g/L NaCl, 0.2 g/L MgSO₄·7H₂O, 10 mg/L CaCl₂·10 mg/L FeSO₄·7H₂O, 0.6 mg/L NiSO₄·7H₂O and 2 mL/L trace metal solution containing 1.68 mg/L Na₂MoO₄·2H₂, 7 mg/L ZnSO₄·7H₂O, 0.5 mg/L CuSO₄·5H₂, 1 mg/mL H₃BO₃, 0.66 mg/L MnSO₄·5H₂O, 1 mg/L CoCl₂·6H₂O. H. marinus strain MH-110 was cultivated as 50 mL cultures in 300 mL Erlenmeyer baffled flasks, or in small culture volume (1 mL) in 24-well cell culture plates using a gaseous atmosphere containing H₂, CO₂ and O₂. To prevent cells from forming aggregates, a glass bead of ca. 3 mm diameter was added to each 1 mL culture. The Erlenmeyer flask, or the 24-well plate, respectively, was then placed into a 2 L anaerobic jar (anaerobic jar “small”, Cat.-No. 3.380.202, Schuett-Biotec GmbH, Germany) which was filled with a gaseous atmosphere of H₂, CO₂ and O₂ at different ratios (H₂:CO₂:O₂:N₂=87:10:3:0; H₂:CO₂:O₂:N₂=88:10:2:0 and H₂:CO₂:O₂:N₂=3:10:10:77). The jar was placed in an incubator set at 37° C., 150 rpm. The atmosphere was refreshed every 24 hours. The optical density of the cultures was measured using a photometer set to a wavelength of 540 nm (OD₅₄₀).

Results: Growth of H. marinus strain MH-110 was observed in all of the tested atmospheres. It was found however that the atmosphere H₂:CO₂:O₂:N₂=88:10:2:0 generated more biomass in a shorter time compared to cultures cultivated in a low-hydrogen atmosphere of H₂:CO₂:O₂:N₂=3:10:10:77.

### Example 2: Species Validation of H. marinus by Colony PCR and 16S rDNA Sequencing

H. marinus MH-110 was grown in a medium as set out above in Example 1 which was solidified by the addition of 1.5% agar-agar (powder, BioScience, BioScience Grade, Carl Roth). For the culturing on plates, the atmosphere was set to H₂:CO₂:O₂:N₂=88:10:2:0. H. marinus colonies from agar plates were picked and subjected to colony PCR using primers which amplify indicative parts of genes encoding bacterial 16S rRNAs.

For that purpose, 32.5 μl double distilled H_{20, 10} μl 5x Phusion Hot Start II DNA Polymerase buffer, dNTP Mix (10 mM each, Thermo Scientific), 2.5 μl of each primer (10 μM stock), 0.5 μl Phusion Hot Start II DNA Polymerase (Thermo Scientific) were mixed in a 200 μl single use plastic PCR tube. Cell material obtained from an agar plate was picked with a sterile pipette tip and transferred to the PCR tube. The PCR was then performed with the following settings: 1 min at 98° C. followed by 35 cycles of 10 sec at 98° C., 20 sec at 53° C., 45 sec at 72° C., followed by 6 min at 72° C. The reaction subsequently was kept at 4° C., prior to further analysis. The following primers were used:

Forward primer (SEQ ID NO: 4):
5′-AGAGTKTGATCMTGGCTCAG-3
Reverse primer (SEQ ID NO: 5):
5′-GACGGGCGGTGTGTRCA-3′.

Primer mixtures with variable positions were used. The primers do not exactly match their corresponding target DNA which renders them useful for generating PCR products from a variety of bacterial species especially, including yet unknown species. The parts of the 16S IRNA genes which are PCR-amplified by using these primers are considered to be indicative for a specific bacterial species (K=G or T: M=A or C: R=A or G). The resulting PCR product was sequenced with the above primers, and the sequence of the PCR product was determined. The sequence of the PCR product was analyzed using the Nucleotide Basic Local

Alignment Search Tool (BLASTN) and the nucleotide collection (nr/nt) provided by the National Center for Biotechnology Information (NCBI). The BLASTN algorithm identifies regions of local similarity between sequences.

Results: The amplified DNA fragment (1402 nucleotides) showed 100% percent sequence identity to the 16S rRNA gene of H. marinus. This was understood as evidence that the colony was a H. marinus colony. The nucleotide sequence of the 16S rRNA gene of H. marinus is depicted in SEQ ID NO:6.

### Example 3: Construction of a sf-GFP-producing Strain of H. marinus

All expression plasmids used in this experiment contain the pAM5409 backbone (Bishé, et al., 2019). A GFP expression cassette was created, consisting of a gene encoding a streptagged superfolder (sf)-GFP (Pédelacq et al., 2006) driven by the synthetic promoter BBa_J23119, combined with the ribosomal binding site (RBS) BBa_B0034 (both derived from the iGEM Registry of Standard Biological Parts), and terminated by the terOOP terminator sequence amplified from the pUR_dualTag plasmid obtained from Prof. Wilde (University of Freiburg, Germany). The nucleotide sequence of the sf-GFP-expression cassette is set out herein as SEQ ID NO:7. The sequence of the sf-GFP-expression cassette was cloned into the pAM5409 plasmid backbone resulting in the plasmid pHM38.

Recombinant H. marinus strains were created via parental mating with an E. coli WM3064 donor strain carrying the pAM5409-based expression plasmids. For this purpose, a H. marinus MH-110 culture was inoculated from a cryostock in Basal Medium and cultured under standard autotrophic growth conditions to an OD₅₄₀ of 0.6-1.4 and, subsequently, concentrated to an OD₅₄₀ of 20-30. E. coli WM3064 donor strain carrying plasmid pHM38 was cultured overnight in LB medium supplemented with 0.3 mM diaminopimelic acid (DAP) and 50 μg/mL kanamycin. The culture was washed twice in LB medium +0.3 mM DAP, cells were suspended in LB+0.3 mM DAP and incubated for 15 min at 37° C., and 180 rpm. The culture was then concentrated 10-fold. 100 μL of the concentrated H. marinus and E. coli cultures were mixed and spread on a 0.45 um nitrocellulose filter (Carl Roth, Germany) placed on a Basal Medium 1% agar plate supplemented with 0.3 mM DAP and 5% LB. The plate was incubated under standard autotrophic growth conditions for 16 hours. The conjugation mixture was then washed from the filter using 500 μL of Basal Medium containing 15 μg/ml kanamycin. 100 μL of the suspension was used to inoculate 900 μL Basal Medium containing 15 μg/mL kanamycin and cultured in a 24-well cell culture plate under standard autotrophic growth conditions for approx, three days. The cultures were then diluted 1/10 in fresh Basal Medium containing 15 μg/mL kanamycin and incubated under standard autotrophic growth conditions for two days. Incubation was carried out in the medium and under conditions described in Example 1 for agar plates and 24-well plates. The gas atmosphere was set to H₂:CO₂:O₂:N₂=88:10:2:0.

The conjugation procedure was considered successful when
a) growth was observed in selection medium containing 15 μg/mL kanamycin and the liquid cultures reached an OD₅₄₀ between 0.5 to 1.4,
b) 16S rRNA sequencing of a cell sample confirmed the presence of H. marinus,
c) no growth on LB agar plates was observed (thereby excluding contamination with E. coli or other heterotrophic microorganisms).

The purity of the conjugated H. marinus strains was confirmed by phase contrast light microscopy (100×) and 16S-rRNA sequencing using primers set out above in SEQ ID NO:4 and SEQ ID NO:5. Additionally, the sample was streaked on a LB agar plate and incubated in the presence of air at 37° C. for several days to confirm that the culture was pure of viable E. coli cells.

The liquid cultures derived from the conjugation/selection procedure were analyzed by fluorescence microscopy. The samples were excited using light of a wavelength of 485 nm and the emitted fluorescence was captured at a wavelength of 535 nm. To quantify the sf-GFP fluorescence emitted by the recombinant strains, 100 μL of the cultures were analyzed in a black flat-transparent bottom 96-well plate (Greiner BioOne, Germany) using the GeniusPro® plate reader (Tecan Group, Switzerland) set to 485 nm excitation and 535 nm emission wavelength.

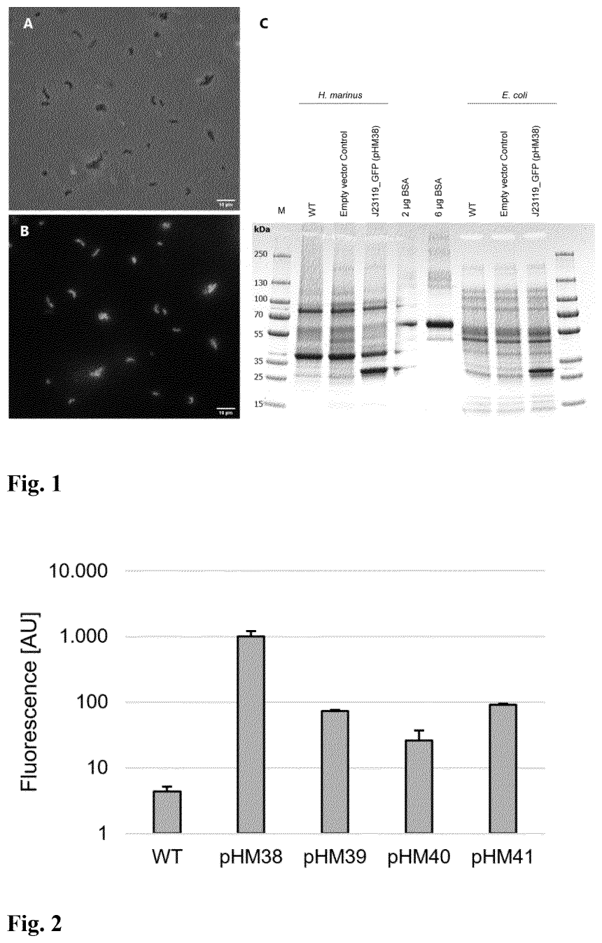

Results: The results are depicted in FIGS. 1A and B. It can be seen that only the H. marinus cells which contained the full reporter gene construct show strong fluorescence.

### Example 4: Analysis of the sf-GFP-Expressing H. marinus Strain by SDS-PAGE

The recombinant H. marinus strain carrying pHM38 (J23119_GFP) was analyzed by polyacrylamide gel electrophoresis in the presence of sodium dodecyl sulfate (SDS-PAGE). The strain was plated on a Basal Medium agar plate supplemented with 15 μg/mL kanamycin and incubated under standard autotrophic growth conditions for five days. The biofilm was collected and resuspended in 400 μL of 1× Buffer W (IBA Lifesciences GmbH. Germany). Approx. 200 μL silica beads (size 0.1-0.2 mm) were added and cells were disrupted via bead beating in a FastPrep device (MP Biomedicals Germany GmbH) using three cycles at 7.5 m/s for 45 sec with 5 min breaks on ice. The cell debris was separated from the soluble fraction via centrifugation at 14.000 rpm and 4° C. for 10 min, and the protein content of the soluble fraction was measured using the standard Bradford method. 16 μg of total protein were mixed with reducing 4× RotiLoad loading buffer (Carl Roth. Germany) and incubated at 95° C. for 15 minutes. Samples were loaded on a NuPAGE™ 4-20% polyacrylamide gel (Thermo Scientific Fisher). PageRuler™ Prestained Protein Ladder was used as protein size reference. Standard SDS-Running Buffer (final concentration: 0.25 M Tris-base, 1.924 M Glycine.

0.347 M SDS) was used. SDS-PAGE was performed at 100 V for 20 min followed by 200 V for ca. 30-60 min. The resulting gel was stained with PageBlue™ Protein Staining Solution (Thermo Scientific) overnight at room temperature. The gel was washed with de-ionized water until protein bands became distinguishable.

Results: The results are depicted in Fig. IC. When compared to the wild-type control the recombinant H. marinus strain containing the full reporter gene construct produced an additional protein band corresponding to a signal in the SDS gel at around 26 kDa. The corresponding protein band was excised. Subsequently, the resulting peptides were subjected to analysis by mass spectrometry. The masses of the resulting peptides exactly matched the expected masses of an in silico digested sf-GFP. Thus, the results show that sf-GFP is produced by the recombinant H. marinus strain. Staining of the gel with Coomassie R 250 brilliant blue revealed that the recombinant protein (sf-GFP) was produced by the recombinant cells in comparably large amounts. The amount of sf-GFP in the soluble fraction of the recombinant H. marinus pHM38 cell lysate is comparable to what is present in an E. coli strain containing a similar construct. When comparing the total soluble protein amount of the bovine serum albumin (BSA) standard to the total soluble protein amount of sf-GFP detected in a cell-free extract of e.g. H. marinus pHM38, it is estimated that heterologous sf-GFP represents about 18-31% of the total protein in the soluble fraction of the recombinant H. marinus strain. This can be estimated upon SDS-PAGE and Coomassie Brilliant Blue R-250 staining.

### Example 5: Construction of sf-GFP-Expressing Strains using Synthetic Promoter Variants

To examine whether the expression levels can be modulated, different synthetic promoters and ribosomal binding sites were combined. The promoter sequences J23119 (SEQ ID NO:8), J23111 (SEQ ID NO:9) and J23114 (SEQ ID NO: 10) were derived from the iGEM parts registry and combined with different ribosomal binding sites. The ribosomal binding site sequences named BBa_B0034 (SEQ ID NO:11), BBa_B0064 (SEQ ID NO:12) and BBa_B0031 (SEQ ID NO:13) were also derived from the iGEM parts registry. Moreover, a ribosomal binding site derived from the riboflavin biosynthetic gene B. subtilis ribDG was used (SEQ ID NO:14).

The GFP-expression plasmid (pHM38) described in Example 3 was used. To create variants containing different promoters and RBSs, pHM38 was digested using KpnI/NheI. The oligonucleotides DNA_039 (SEQ ID NO:15)/DNA_040 (SEQ ID NO:16), containing the J23111 promoter and the BBa_B0064 RBS, were annealed and ligated into the digested backbone, to generate plasmid pHM39. The oligonucleotides DNA_041 (SEQ ID NO:17)/DNA_042 (SEQ ID NO: 18), containing the J23114 promoter and the BBa_B0031 RBS, were annealed and ligated into the digested backbone, to generate plasmid pHM40. The oligonucleotides DNA_043 (SEQ ID NO:19)/DNA_044 (SEQ ID NO:30), containing the J23119 promoter and the RBS derived from the B. subtilis ribDG gene, were annealed and ligated into the digested backbone, to generate plasmid pHM41. The sequence of pHM38, pHM39, pHM40 and pHM41 are depicted as SEQ ID NO:22, 23, 24 and 25, respectively.

H. marinus wild-type strain was conjugated with the plasmids pHM38-pHM41, respectively, using the conjugation method described above. H. marinus strains carrying the respective plasmids were inoculated to an OD₅₄₀ of 0.01 and cultured under standard autotrophic growth conditions for four days. sf-GFP fluorescence was measured using the GeniusPro™ plate reader.

Results: The results are depicted in FIG. 2 and in the below Table 1. The data show elevated sf-GFP levels for all recombinant H. marinus strains in comparison to the wild type. The data suggest that depending on the choice of the promoter-RBS variant, a low level of expression, a medium level of expression or a high level of expression of the heterologous gene occurred.

TABLE 1
Fluorescence of sf-GFP-expressing H. marinus
strains in comparison to the wild-type (WT)

| | | | sf-GFP- | |
|---|---|---|---|---|
| | Expected promoter | Expected RBS | fluorescence | |
| Strain | strength | strength | fold-change | Stdev |
| WT | — | — | 1 | n.a. |
| pHM38 | Strong | Strong | 227 | 47.2 |
| pHM39 | Moderate | Moderate | 17 | 0.6 |
| pHM40 | Weak | Weak | 6 | 2.6 |
| pHM41 | Strong | Weak | 21 | 0.9 |

It is thus possible to achieve different production levels of a certain heterologous protein depending on the combination of the genetic elements.

### Example 6: Overexpression of Human Somatotropin (rhGH)

H. marinus MH-110 was also modified to produce recombinant human somatotropin (rhGH). The sequence of the hGH-encoding gene was codon-optimized for E. coli employing the ATGme online software (Daniel et al. 2015). A nucleotide sequence specifying an N-terminal 6xHis-tag and a TEV protease cleavage site were added to the gene sequence. The resulting DNA sequence including BcuI/BamHI restriction sites (SEQ ID NO:3) was purchased from Integrated DNA Technologies, Inc. The BcuI/BamHI-digested hGH DNA-fragment was cloned into the BcuI/BamHI-digested pHM39 plasmid to generate pHM64. In this plasmid, hGH is under the control of the promoter J23111 (SEQ ID NO:9) and RBS Bba_B0064 (SEQ

ID NO: 12).

To generate an empty control plasmid, a small fragment from pHM38 containing the Strep tag and the nucleotides coding for the first eight amino acids of sf-GFP were amplified from pHM38 using primers DNA_174 and DNA_53.

DNA_174 (SEQ ID NO: 28):
ATAATGAATTCTATAGGTACCATAAA-
TAAGGTTTTCCCCTGATGCTGCCACGCGTGACTAGTATGTGGAGCCACCC
GCAG
DNA_053 (SEQ ID NO: 29):
TTTGGTCTCAAGCTTGGATCCAGTGAAAAGTTCTTCTCC

These primers put a scrambled DNA sequence with the same length of the J23111 promoter in front of the gene. The scrambled fragment has no promoter function and therefore serves as negative control for ‘no gene expression’. The fragment was cut via EcoRI BamHI and cloned into the similarly cut pHM38 to generate pHM42.

Proteins were extracted from the soluble and insoluble fractions of E. coli and H. marinus cell lysates from strains carrying either the pHM64 (SEQ ID NO:27) plasmid coding for hGH under control of a moderate promoter-RBS combination or an empty vector control (pHM42, SEQ ID NO:26). The extracted proteins of both E. coli and H. marinus rhGH expressing strains were separated via SDS-PAGE and subjected to Western Blot analysis. The Nterminal His-tag of rhGH was employed for immunological detection. Proteins were separated via SDS page as described above. A Low-Fluorescence PVDF Transfer Membrane (0.2 μm, Thermo Scientific™) was activated in methanol for 15 sec and washed in ddH₂O for 2 min followed by an equilibration step in Towbin buffer (25 mM Tris, 192 mM Glycine and 25% (v/v) methanol) for 15 min. Proteins were transferred to the membrane via a semi-dry transfer system (Trans-Blot SD Semi-Dry Transfer Cell, Biorad) with Towbin buffer at 50 mA for 1 hr. The membrane was blocked in TBST+5% BSA (20 mM Tris base, 150 mM NaCl, 0.1% Tween 20 (v/v), 5% BSA (w/v)) for 1 hr at 4° C. The 6x-His Tag Monoclonal Antibody (HIS.H₈), HRP (Thermo Fisher Scientific) was diluted 1/1000 in TBST +5% BSA and the membrane was probed overnight at 4° C. The membrane was washed three times in TBST for 10 minutes. 20 mL freshly prepared staining solution (200 mg/L diaminobenzidine (DAB), 100 mg/L CuSO₄, 5 mg/L NiSO₄x6H₂O, 0.6% H₂O₂) were added to the membrane and incubated at room temperature till protein bands became visible. The reaction was stopped by washing the membrane in ddH₂O.

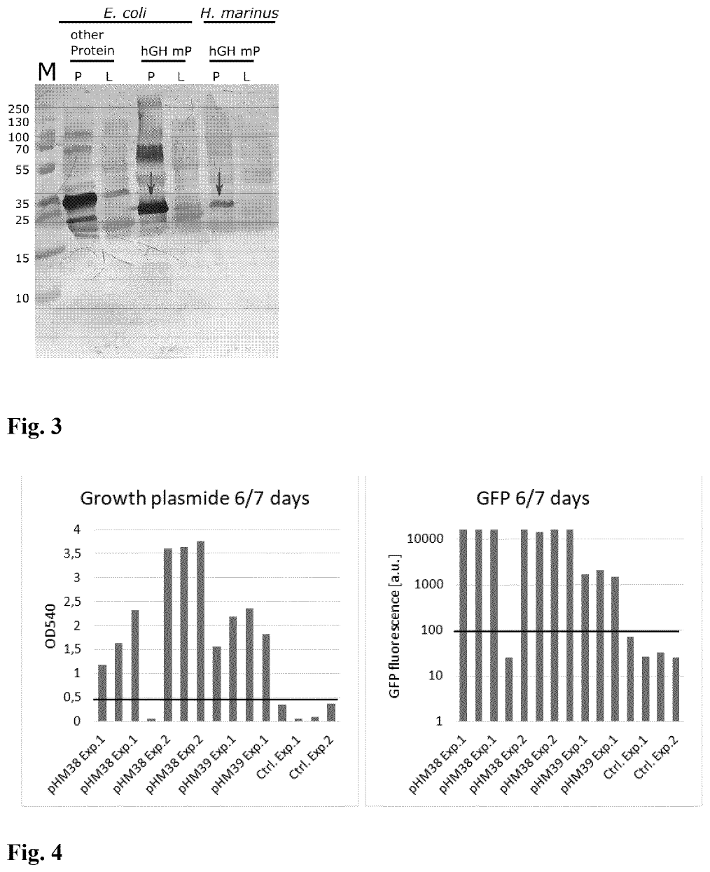

Results: The results of the immune detection are set out in FIG. 3. The results revealed a strong band at the size corresponding to rhGH for the E. coli protein extract. A band at the same height was detected in the insoluble fraction of the H. marinus protein extract indicating that both strains produced the hormone.

### Example 7: Transformation of H. marinus with Replicative Plasmid DNA via Electroporation

H. marinus strain MH-110 was cultured as 50 mL cultures in 300 mL Erlenmeyer baffled flasks using the Basal Medium described in Example 1 above. The Erlenmeyer flask was placed into a 2 L anaerobic jar (anaerobic jar “small”, Cat. No. 3.380.202, Schuett-Biotec GmbH, Germany) which was filled with a gaseous atmosphere of H₂, CO₂ and O₂ (at the ratio (H₂:CO₂:O₂:N₂=87:10:3:0). The jar was placed in an incubator set at 37° C., 150 rpm. The culture was grown for 16 hrs. The culture was then diluted to an OD₅₄₀=0.04-0.06 into 50 mL fresh media and cultivation under the above recited conditions was continued for 4-5 hrs.

Cells were harvested by centrifugation for 10 min at 4000× g. 16° C., and cells were concentrated to an OD₅₄₀=5. The concentrated cell suspension was centrifuged at 5000× g for 5 min and the cell pellet was resuspended in an equal volume of IM Ectoine (Sigma-Aldrich, 81619-10G-F) solution. The centrifugation and resuspension steps were repeated twice (3-times in total). 100 μl of the cell suspension were mixed with 300 ng plasmid DNA (pHM38 or pHM39) in a 2 mm gap electroporation cuvette (VWR, 732-1136). Electroporation was conducted at 1.8 kV, 200 Ohm, 25 μF with a Gene Pulser II (Biorad) controlled by a Pulse Controller Pus (Biorad).

Immediately after electroporation, 900 μl of Basal Medium, incubated in an atmosphere of H₂:CO₂=96.7:3.3 for 4 hrs at 4-16° C., was added to the cells and the culture was transferred into the wells of a 24-well microtiterplate, containing a 5 mm glass bead. During the whole procedure starting with the first centrifugation step, cells, media, microtiterplate and cuvettes were kept at low temperature (4-16° C.). The microtiterplate was placed in an anaerobic jar and atmosphere was exchanged for a mixture of air, CO₂ and H₂ in a ratio of 90:7:3. Cultures were incubated for 3 hrs at 37° C., 150 rpm. Cells were then harvested by centrifugation (5.000× g. 3 min at room temperature) and resuspended in Basal Medium containing 15 μg/ml kanamycin (Selection Medium). The resulting cultures were diluted 1/10 into Selection Medium. Both undiluted and diluted cultures were transferred to a 24-well microtiterplate, containing a 5 mm glass bead per well. Cultures were incubated for 6 to 7 days in an atmosphere of H₂:CO₂: O2=87:10:3 at 37° C., 150 rpm. Gas atmosphere was exchanged after 3 days. Culturing was continued for 7 days.

Results: The results are depicted in FIG. 4. After 6 to 7 days of incubation, most cultures showed growth and GFP fluorescence. A transformation was considered successful, when the culture showed growth and GFP fluorescence compared to the controls. The experiment was repeated twice with 5-6 replicates each. In the first experiment 6 of 6 and in the second experiment 4 of 5 transformations were successful, resulting in an overall transformation efficiency of 90 +10%. None of the controls showed growth or GFP fluorescence which proves that (i) the electroporation step is required for H. marinus to take up the DNA and that (ii) H. marinus is most likely not naturally competent under these cultivation conditions. Additional experiments demonstrated that successful transformation was achieved with ectoine concentrations ranging from 0.25-2 M.

### Example 8: Transformation of H. marinus with Integrative DNA via Electroporation

H. marinus strain MH-110 was cultured and prepared for electroporation as described in Example 7. For integration, a linear DNA fragment was used consisting of a kanamycin resistance cassette (Kan^{R}) and GFP under the control of a strong constitutive promoter (J23119_GFP) and flanked by two homologous sequences of approx. 600 bp that target integration into the bla locus of the H. marinus genome.

100 μl of the cell suspension in 1 M Ectoine were mixed with 700 ng DNA in a 2 mm gap electroporation cuvette (VWR, 732-1136). Electroporation and the recovery step were performed as described in Example 7. After 3 hrs of recovery in Basal Medium, 450 μl of the cell cultures were harvested by centrifugation (5.000× g, 3 min at room temperature) and resuspended in 1 mL Selection medium containing 5 μg/ml kanamycin. The resulting cultures were transferred to a 24-well microtiter plate, containing a 5 mm glass bead per well. Cultures were incubated for 3 days in an atmosphere of H₂:CO₂:O₂=87:10:3 at 37° C., and 150 rpm. The cultures were harvested by centrifugation (5.000× g, 3 min at room temperature) and resuspended in Selection medium containing 15 μg/ml kanamycin. Cultures were grown for additional 3 to 5 days (6 to 8 days in total) in an atmosphere of H₂:CO₂:O₂=87:10:3 at 37° C., 150 rpm. Gas atmosphere was exchanged after 3 days.

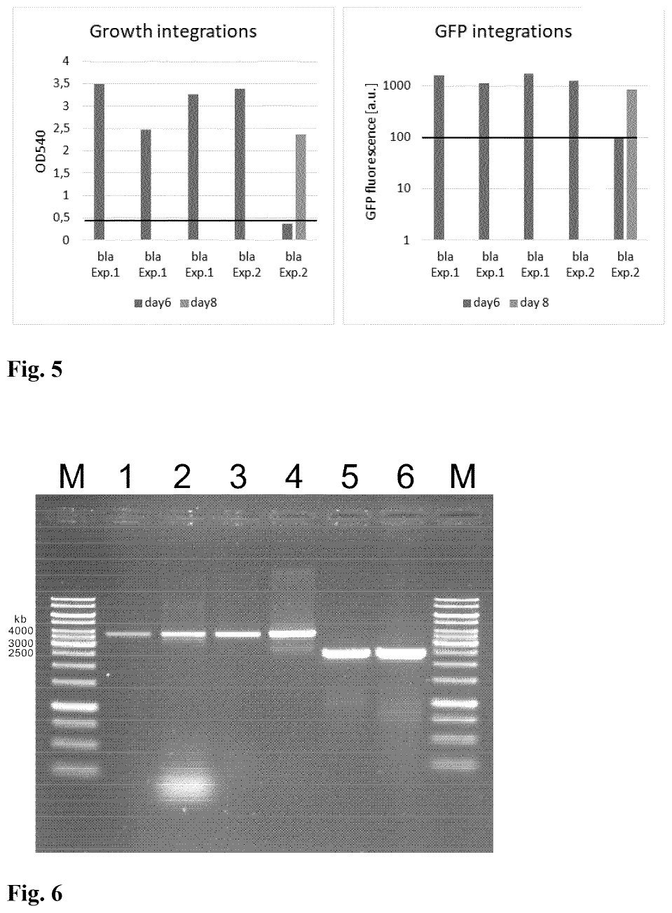

Results: The results are depicted in FIG. 5. After 6 to 8 days of incubation, all cultures showed growth and GFP fluorescence. The experiment was repeated twice with 2-3 replicates each. In both experiments all transformation replicates were successful resulting in an overall transformation efficiency of 100%.

Integration of the gene expression cassettes was furthermore confirmed for four transformants by colony PCR using primers that bind within the genome of H. marinus outside the DNA sequence used to flank the integration cassettes. The results are shown in FIG. 6. Successful integration results in a PCR fragment of about 3.9 kb, whereas wild type untransformed H. marinus or H. marinus carrying only replicative plasmids results in a PCR fragment of about 2.5 kb. All tested transformants showed the expected larger PCR fragment.

### LITERATURE

1. Nishihara, H., Igarashi, Y., and Kodama, T. (1989) Archives of Microbiology 152, 39-43
2. Arai, H., and Ishii, M. (2019) Microbiol Resour Announc 8
3. Jo, B. H., Hwang, B. H., and Cha, H. J. (2014) J Biotechno/185, 37-38
4. Toyoda, K., Ishii, M., and Arai, H. (2018) J Biosci Bioeng 126, 730-735
5. Toyoda, K., Yoshizawa, Y., Arai, H., Ishii, M., and Igarashi, Y. (2005) Microbiology 151, 3615-3625
6. Hoff, B., Plassmeier, J., Blankschien, M., Letzel, A. C., Kourtz, L., Schroder, H., Koch, W., and Zelder, O. (2021) Angew Chem Int Ed Engl 60, 2258-2278
7. Kachel, B., and Mack, M. (2020) Metab Eng 7.
8. Nishihara, H., Igarashi, Y., and Kodama, T. (1991) Journal of fermentation and bioengineering 72, 358-361
9. Nishihara, H., Miyashita, Y., Aoyama, K., Kodama, T., Igarashi, Y., and Takamura, Y. (1997) Biochem Biophys Res Commun 232, 766-770
10. Nishihara, H., Miyata, Y., Miyashita, Y., Bernhard, M., Pohlmann, A., Friedrich, B., and Takamura, Y. (2001) Biosci Biotechnol Biochem 65, 2780-2784
11. Nishihara, H., Yaguchi, T., Chung, S. Y., Suzuki, K., Yanagi, M., Yamasato, K., Kodama, T., and Igarashi, Y. (1998) Arch Microbiol 169, 364-368
12. Kim, J. Y., Jo, B. H., and Cha, H. J. (2011) J Biotechnol 155, 312-319
13. Bacher, A., Eberhardt, S., Eisenreich, W., Fischer, M., Herz, S., Illarionov, B., Kis, K., and Richter, G. (2001) Vitam Horm 61, 1-49
14. Fischer, M., and Bacher, A. (2005) Nat Prod Rep 22, 324-350
15. Joosten, V., and van Berkel, W. J. (2007) Curr Opin Chem Biol 11, 195-202
16. Stahmann, K. P., Revuelta, J. L., and Seulberger, H. (2000) Appl Microbiol Biotechnol 53,509-516
17. Schwechheimer, S. K., Becker, J., Peyriga, L., Portais, J. C., Sauer, D., Muller, R., Hoff, B., Haefner, S., Schroder, H., Zelder, O., and Wittmann, C. (2018) Metab Eng 47, 357-373
18. Abbas, C. A., and Sibirny, A. A. (2011) Microbiol Mol Biol Rev 75, 321-360
19. Schwechheimer, S. K., Park, E. Y., Revuelta, J. L., Becker, J., and Wittmann, C. (2016) Appl Microbiol Biotechnol 100, 2107-2119
20. Averianova, L. A., Balabanova, L. A., Son, O. M., Podvolotskaya, A. B., and Tekutyeva, L. A. (2020) Front Bioeng Biotechnol 8, 570828
21 Perkins, J., and Pero, J. (2002) Biosynthesis of riboflavin, biotin, folic acid, and cobalamin, in Bacillus subtilis and Its Closest Relatives: from Genes to Cells (Sonenshein, A., Hoch, J., and Losick, R, eds.), ASM Press, Washington DC, pp 271-286
22. Acevedo-Rocha, C. G., Gronenberg, L. S., Mack, M., Commichau, F. M., and Genee, H. J. (2018) urr Opin Biotechnol 56, 18-29
23. Mack, M., van Loon, A. P., and Hohmann, H. P. (1998) J Bacteriol 180, 950-95
24. Winkler, W. C., Cohen-Chalamish, S., and Breaker, R. R. (2002) Proc Natl Acad Sci USA 99, 15908-15913
25. Mironov, A. S., Gusarov, I., Rafikov, R., Lopez, L. E., Shatalin, K., Kreneva, R. A., Perumov, D. A., and Nudler, E. (2002) Cell 111, 747-756
26 Pedrolli, D. B., Kuhm, C., Sevin, D. C., Vockenhuber, M. P., Sauer, U., Suess, B., and Mack, M. (2015) Proc Natl Acad Sci U S A 112, 14054-14059
27. Haase, I., Grawert, T., Illarionov, B., Bacher, A., and Fischer, M. (2014) Methods Mol Biol 1146, 15-40
28. Hemberger, S., Pedrolli, D. B., Stolz, J., Vogl, C., Lehmann, M., and Mack, M. (2011) BMC Biotechnol 11, 119
29. Schwarz, J., Konjik, V., Jankowitsch, F., Sandhoff, R., and Mack, M. (2016) Angew Chem Int Ed Engl 55, 6103-6106
30. Pedrolli, D. B., Jankowitsch, F., Schwarz, J., Langer, S., Nakanishi, S., and Mack, M. (2014) Methods Mol Biol 1146, 41-63
31. Jankowitsch, F., Schwarz, J., Ruckert, C., Gust, B., Szczepanowski, R., Blom, J., Pelzer, S., Kalinowski, J., and Mack, M. (2012) J Bacteriol 194, 6818-6827
32. Haase, I., Sarge, S., Illarionov, B., Laudert, D., Hohmann, H. P., Bacher, A., and Fischer, M. (2013) Chembiochem 14, 2272-2275
33. Sarge, S., Haase, I., Illarionov, B., Laudert, D., Hohmann, H. P., Bacher, A., and Fischer, M. (2015) Chembiochem 16, 2466-2469
34. Olson, K., Fenno, J., Lin, N, et al. (1981) Nature 293, 408-411
35. Bishe, B., Arnaud, T., and Golden, J. W. (2019), Iscience 20, 216-228
36. Daniel E., Onwukwe, G. U., Wierenga, R. K., Quaggin S. E., Vainio S. J, and Krause, M. (2015), BMC Bioinformatics, 16, Article number: 303

1. Chemolithoautotrophic bacterium of the genus Hydrogenovibrio which has been genetically modified to produce one or more heterologous proteins.

2. Chemolithoautotrophic bacterium of claim 1, wherein the chemolithoautotrophic bacterium is of the species Hydrogenovibrio marimis.

3. Chemolithoautotrophic bacterium of claim 2, wherein the chemolithoautotrophic bacterium is derived from Hydrogenovibrio marinus MH-110, deposited at the Japanese Cell Collection (JCM) under deposition number JCM 7688.

4. Chemolithoautotrophic bacterium of claim 1, wherein the chemolithoautotrophic bacterium has been genetically modified to produce a mammalian protein.

5. Chemolithoautotrophic bacterium of claim 4, wherein the mammalian protein is a therapeutically active protein.

6. Chemolithoautotrophic bacterium of claim 5, wherein the mammalian protein is human somatotropin (hGH) or a biologically active fragment thereof.

7. Chemolithoautotrophic bacterium of claim 6, wherein the human somatotropin (hGH) comprises or consists of
(a) the amino acid sequence of SEQ ID NO:1, or
(b) an amino acid sequence having at least 80% sequence identity to the amino acid sequences of SEQ ID NO:1.

8. Chemolithoautotrophic bacterium of claim 1, wherein the chemolithoautotrophic bacterium comprises a plasmid.

9. Chemolithoautotrophic bacterium of claim 6, wherein the plasmid is a non-integrating plasmid.

10. Method for the production of one or more heterologous proteins in a host cell, comprising
(a) providing a chemolithoautotrophic bacterium of claim lany of claims 1 9;
(b) culturing the host cell under conditions that allow the production of the heterologous protein;
(c) obtaining the heterologous protein.

11. Method of claim 10, wherein step (b) comprises culturing the host cell in one of the following gas mixtures
(a) H₂:CO₂:O₂:N₂=89:10:1:0, 88:10:2:0, 87:10:1:0, or
(b) H₂:CO₂:O₂:N₂=77:10:10:3, 78:10: 10:2, and 79:10:10:.

12. Method of claim 10, wherein step (c) comprises disruption of the cells.

13. Method of claim 10, wherein step (c) further comprises purification of the heterologous protein.

14. Use of the chemolithoautotrophic bacterium of claim 1 as a host cell for the production of a heterologous protein.

15. Kit for the production of a heterologous protein comprising the chemolithoautotrophic bacterium of claim 1.

16. Method of introducing an exogenous nucleic acid molecule into a chemolithoautotrophic bacterium, and preferably a bacterium of the genus Hydrogenovibrio, said method comprising:
a) preparing a suspension of bacterial cells, preferably bacterial cells of the genus Hydrogenovibrio, in a fluid that comprises ectoine and an exogenous nucleic acid molecule, and
b) electroporating the bacterial cells to introduce the exogenous nucleic acid molecule in the cells.

17. Method of claim 17, wherein the ectoine is present in the fluid in a concentration of 0.25 to 2.5 mol/L, and preferably 0.5 to 2.0 mol/L.

18. Method of claim 17 er 18, wherein the method further comprises:
c) after electroporation, culturing the bacterial cells in a culture medium that was kept in an atmosphere that essentially consists of H₂ and CO₂ for at least 2 hours before addition to the cells.

19. Method of claim 19, wherein the atmosphere essentially consists of 96.7% H₂ and 3.3% CO₂.

20. Method of claim 16, wherein the exogenous nucleic acid molecule is a DNA molecule, and preferably a linear DNA molecule or a circular DNA plasmid or vector.


SEQUENCE LISTING (32 sequences)

SEQ ID NO: 1 - protein, 217 aa, Homo sapiens
  REGION 1..217: note = human growth hormone (hGH)
MATGSRTSLLLAFGLLCLPWLQEGSAFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 2 - protein, 231 aa, synthetic construct
  REGION 1..231: note = hGH with His-tag
MHHHHHHENLYFQSMATGSRTSLLLAFGLLCLPWLQEGSAFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 3 - DNA, 745 bp, synthetic construct
  misc_feature 1..745: note = hGH, codon-adapted for E. coli with His-Tag
  source 1..745: mol_type = other DNA
gtaaaacgacggccagtgactagtatgcaccatcaccatcaccatgaaaacctttacttccagagcatggcgactgggtcccgcacttcattgttgctggcatttggacttctttgtctgccttggttgcaggaggggagtgcttttcccactataccattatctcgtctgttcgacaacgcgatgttacgggctcacagacttcaccaattagcttttgatacataccaagagtttgaggaagcctatataccaaaggaacaaaagtattcatttcttcaaaatccgcaaaccagtctttgcttcagcgagagtataccaacaccgtccaaccgtgaggaaacacaacagaagagcaacttagaacttcttcgtatttctcttttgttaatacagtcatggttggaacccgtgcagttcctgagatctgtttttgccaattcactggtctacggtgcatccgatagtaatgtgtacgatttattgaaggacttggaagagggcatccagacgttgatgggtcggcttgaagacggttcgcccagaactggccaaatatttaagcaaacatattccaagttcgacacaaattctcataatgacgatgcactgttgaagaactatggtcttttatactgtttccgcaaggatatggataaagtggaaacatttctgcgtattgtccagtgtcgctcggttgaaggttcctgcggattttagggatcccatggtcatagctgtttcc

SEQ ID NO: 4 - DNA, 20 bp, synthetic construct
  misc_feature 1..20: note = Primer
  variation 6: note = k is g or t
  variation 12: note = m is a or c
  source 1..20: mol_type = other DNA
agagtktgatcmtggctcag

SEQ ID NO: 5 - DNA, 17 bp, synthetic construct
  misc_feature 1..17: note = Primer
  variation 15: note = r is a or g
  source 1..17: mol_type = other DNA
gacgggcggtgtgtrca

SEQ ID NO: 6 - DNA, 1402 bp, Hydrogenovibrio marinus
  misc_feature 1..1402: note = 16S rRNA
  source 1..1402: mol_type = genomic DNA
agagtttgatcctggctcagaatgaacgctggcggcaggcttaacacatgcaagtcgaacggtaacaggagaaagcttgctttcttgctgacgagtggcggacgggtgagtaatgtatgggaatctgccctctagttggggacaacatatggaaacgtatgctaataccgaatgtgatctacggattaaaggtgccctctccttggaaggtatcgctagaggatgagcccatattagattagctagttggtagggtaaaggcctaccaaggcgacgatctatagctggtttgagaggatgatcagccacactgggactgagacacggcccagactcctacgggaggcagcagtggggaatattgcacaatgagggaaaccttgatgcagccatgccgcgtgtgtgaagaaggcctgagggttgtaaagcactttcaattgtgaggaaagctaagtagttaatacctgcttagtgtgacgttaactttagaagaagcaccggctaactctgtgccagcagccgcggtaatacagagggtgcaagcgttattcggaattactgggcgtaaagcgcgcgtaggcggactgttaagtcggttgtgaaagccctgggctcaacctaggaattgcatccgatactggcaatctagagtttaagagagggaaggggaattccaggtgtagcagtgaaatgcgtagatatctggaggaacatcagtggcgaaggcgccttcctggcttaaaactgacgctgaggtgcgaaagcgtgggtagcgaacgggattagataccccggtagtccacgccgtaaacgatgtcaactagctgttggccttattaaaaaggttagtagcgcagctaacgcgataagttgaccgcctggggagtacggtcgcaagattaaaactcaaaggaattgacgggggcccgcacaagcggtggagcatgtggtttaattcgatgcaacgcgaagaaccttaccatcccttgacatcctgcgaactttctagagatagattggtgccttcgggaacgcagtgacaggtgctgcatggctgtcgtcagctcgtgtcgtgagatgttgggttaagtcccgcaacgagcgcaacccttatctttagttgctaccatttagttgagaactctaaagagactgccggtgataaaccggaggaaggcggggacgacgtcaagtcatcatggcccttatgggatgggctacacacgtgctacaatggggggtacaaagagcagctagctggcgacagtgcgcgaatctcataaaacccttcgtagtccggattggagtctgcaactcgactccatgaagtcggaatcgctagtaatcgtgaatcagaatgtcacggtgaatacgttcccgggccttgtacacaccgcccgtc

SEQ ID NO: 7 - DNA, 886 bp, synthetic construct
  misc_feature 1..886: note = GFP expression cassette
  source 1..886: mol_type = other DNA
tctagataatgaattctataggtaccttgacagctagctcagtcctaggtataatgctagcactagagaaagaggagaaatactagtatgtggagccacccgcagttcgaaaaaggatcaagcaaaggagaagaacttttcactggagttgtcccaattcttgttgaattagatggtgatgttaatgggcacaaattttctgtccgtggagagggtgaaggtgatgctacaaacggaaaacttacccttaaatttatttgcactactggaaaactacctgttccatggccaacacttgtcactactctcacttatggtgttcaatgcttttcccgttatccggatcatatgaaacggcatgactttttcaagagtgccatgcccgaaggttatgtacaggaacgcactatatctttcaaagatgacgggacttacaagacgcgtgctgaagtcaagtttgaaggtgatacccttgttaatcgtatcgagttaaaaggtattgattttaaagaagatggaaacattctcggacacaaacttgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtctacacaatctgtcctttcgaaagatcccaacgaaaagcgtgaccacatggtccttcttgagtttgtaactgctgctgggattacacatggcatggatgagctctacaaataaggatcctgaagatccaacgctcggttgccgccgggcgttttttattctgcag

SEQ ID NO: 8 - DNA, 35 bp, synthetic construct
  misc_feature 1..35: note = Promoter J23119
  source 1..35: mol_type = other DNA
ttgacagctagctcagtcctaggtataatgctagc

SEQ ID NO: 9 - DNA, 35 bp, synthetic construct
  misc_feature 1..35: note = Promoter J23111
  source 1..35: mol_type = other DNA
ttgacggctagctcagtcctaggtatagtgctagc

SEQ ID NO: 10 - DNA, 35 bp, synthetic construct
  misc_feature 1..35: note = Promoter J23114
  source 1..35: mol_type = other DNA
tttatggctagctcagtcctaggtacaatgctagc

SEQ ID NO: 11 - DNA, 28 bp, synthetic construct
  misc_feature 1..28: note = RBS BBa_B0034
  source 1..28: mol_type = other DNA
tctagagaaagaggagaaatactagatg

SEQ ID NO: 12 - DNA, 28 bp, synthetic construct
  misc_feature 1..28: note = RBS BBa_B0064
  source 1..28: mol_type = other DNA
tctagagaaagaggggaaatactagatg

SEQ ID NO: 13 - DNA, 30 bp, synthetic construct
  misc_feature 1..30: note = RBS BBa_B0031
  source 1..30: mol_type = other DNA
tctagagtcacacaggaaacctactagatg

SEQ ID NO: 14 - DNA, 44 bp, synthetic construct
  misc_feature 1..44: note = RBS_ribDG
  source 1..44: mol_type = other DNA
actagagacggtaaataacaaaagaggggagggacactagtatg

SEQ ID NO: 15 - DNA, 57 bp, synthetic construct
  misc_feature 1..57: note = Primer DNA_039
  source 1..57: mol_type = other DNA
cttgacggctagctcagtcctaggtatagtgctagcactagagaaagaggggaaata

SEQ ID NO: 16 - DNA, 65 bp, synthetic construct
  misc_feature 1..65: note = Primer DNA_040
  source 1..65: mol_type = other DNA
ctagtatttcccctctttctctagtgctagcactatacctaggactgagctagccgtcaaggtac

SEQ ID NO: 17 - DNA, 58 bp, synthetic construct
  misc_feature 1..58: note = Primer DNA_041
  source 1..58: mol_type = other DNA
ctttatggctagctcagtcctaggtacaatgctagcactagagtcacacaggaaacca

SEQ ID NO: 18 - DNA, 66 bp, synthetic construct
  misc_feature 1..66: note = Primer DNA_042
  source 1..66: mol_type = other DNA
ctagtggtttcctgtgtgactctagtgctagcattgtacctaggactgagctagccataaaggtac

SEQ ID NO: 19 - DNA, 72 bp, synthetic construct
  misc_feature 1..72: note = Primer DNA_043
  source 1..72: mol_type = other DNA
cttgacagctagctcagtcctaggtataatgctagcactagagacggtaaataacaaaagaggggagggaca

SEQ ID NO: 20 - DNA, 37 bp, synthetic construct
  misc_feature 1..37: note = Primer DNA_051
  source 1..37: mol_type = other DNA
tttggtctcactagtacgttgtgtctcaaaatctctg

SEQ ID NO: 21 - DNA, 37 bp, synthetic construct
  misc_feature 1..37: note = Primer DNA_052
  source 1..37: mol_type = other DNA
tttggtctcaagcttccagatgtatgctcttctgctc

SEQ ID NO: 22 - DNA, 8410 bp, synthetic construct
  misc_feature 1..8410: note = plasmid pHM38
  source 1..8410: mol_type = other DNA
agtcctcgtctcgccctcgaattagcccgcttaatgagcgggctttttttgaattaattctcgcgagctggcacgacaggtttcccgactggaaagcgggcagtgagcgcaacgcaattaatgtaagttagcgcgaattgcaagctggccgacgcgctgggctacgtcttgctggcgttcgggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgctggcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccaccatggcatcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgtcggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatcaccgaagcgcttgaccagcccggccatctcgctgcggtactcgtccagcgccgtgcgccggtggcggctaagctgccgctcggacagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtggtggcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgctgggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggcctgcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgaagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggtgcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccctcgcgagaattaattcagataaaaaaaatccttagctttcgctaaggatgatttctagcgatgaccctgctgattggttcgctgaccgtagactggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgacatcgcgcgcgcgcgcgcggatgctagtacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctattctgaccttgccatcacgactgtgctggtcattaaacgcgtattcaggctgaccctgcgcgctgcgcagggctttattgattccatttttacactgatgaatgttccgttgcgctgcccggattacagatcctctagataatgaattctataggtaccttgacagctagctcagtcctaggtataatgctagcactagagaaagaggagaaatactagtatgtggagccacccgcagttcgaaaaaggatcaagcaaaggagaagaacttttcactggagttgtcccaattcttgttgaattagatggtgatgttaatgggcacaaattttctgtccgtggagagggtgaaggtgatgctacaaacggaaaacttacccttaaatttatttgcactactggaaaactacctgttccatggccaacacttgtcactactctcacttatggtgttcaatgcttttcccgttatccggatcatatgaaacggcatgactttttcaagagtgccatgcccgaaggttatgtacaggaacgcactatatctttcaaagatgacgggacttacaagacgcgtgctgaagtcaagtttgaaggtgatacccttgttaatcgtatcgagttaaaaggtattgattttaaagaagatggaaacattctcggacacaaacttgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtctacacaatctgtcctttcgaaagatcccaacgaaaagcgtgaccacatggtccttcttgagtttgtaactgctgctgggattacacatggcatggatgagctctacaaataaggatcctgaagatccaacgctcggttgccgccgggcgttttttattctgcaggagcagaagagcatacatctggaagctttctagaatcgggggcccccggggggattctagaacaatgga

SEQ ID NO: 23 - DNA, 8410 bp, synthetic construct
  misc_feature 1..8410: note = plasmid pHM39
  source 1..8410: mol_type = other DNA
agtcctcgtctcgccctcgaattagcccgcttaatgagcgggctttttttgaattaattctcgcgagctggcacgacaggtttcccgactggaaagcgggcagtgagcgcaacgcaattaatgtaagttagcgcgaattgcaagctggccgacgcgctgggctacgtcttgctggcgttcgggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgctggcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccaccatggcatcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgtcggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatcaccgaagcgcttgaccagcccggccatctcgctgcggtactcgtccagcgccgtgcgccggtggcggctaagctgccgctcggacagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtggtggcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgctgggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggcctgcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgaagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggtgcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccctcgcgagaattaattcagataaaaaaaatccttagctttcgctaaggatgatttctagcgatgaccctgctgattggttcgctgaccgtagactggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgacatcgcgcgcgcgcgcgcggatgctagtacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctattctgaccttgccatcacgactgtgctggtcattaaacgcgtattcaggctgaccctgcgcgctgcgcagggctttattgattccatttttacactgatgaatgttccgttgcgctgcccggattacagatcctctagataatgaattctataggtaccttgacggctagctcagtcctaggtatagtgctagcactagagaaagaggggaaatactagtatgtggagccacccgcagttcgaaaaaggatcaagcaaaggagaagaacttttcactggagttgtcccaattcttgttgaattagatggtgatgttaatgggcacaaattttctgtccgtggagagggtgaaggtgatgctacaaacggaaaacttacccttaaatttatttgcactactggaaaactacctgttccatggccaacacttgtcactactctcacttatggtgttcaatgcttttcccgttatccggatcatatgaaacggcatgactttttcaagagtgccatgcccgaaggttatgtacaggaacgcactatatctttcaaagatgacgggacttacaagacgcgtgctgaagtcaagtttgaaggtgatacccttgttaatcgtatcgagttaaaaggtattgattttaaagaagatggaaacattctcggacacaaacttgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtctacacaatctgtcctttcgaaagatcccaacgaaaagcgtgaccacatggtccttcttgagtttgtaactgctgctgggattacacatggcatggatgagctctacaaataaggatcctgaagatccaacgctcggttgccgccgggcgttttttattctgcaggagcagaagagcatacatctggaagctttctagaatcgggggcccccggggggattctagaacaatgga

SEQ ID NO: 24 - DNA, 8411 bp, synthetic construct
  misc_feature 1..8411: note = plasmid pHM40
  source 1..8411: mol_type = other DNA
agtcctcgtctcgccctcgaattagcccgcttaatgagcgggctttttttgaattaattctcgcgagctggcacgacaggtttcccgactggaaagcgggcagtgagcgcaacgcaattaatgtaagttagcgcgaattgcaagctggccgacgcgctgggctacgtcttgctggcgttcgggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgctggcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccaccatggcatcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgtcggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatcaccgaagcgcttgaccagcccggccatctcgctgcggtactcgtccagcgccgtgcgccggtggcggctaagctgccgctcggacagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtggtggcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgctgggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggcctgcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgaagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggtgcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccctcgcgagaattaattcagataaaaaaaatccttagctttcgctaaggatgatttctagcgatgaccctgctgattggttcgctgaccgtagactggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgacatcgcgcgcgcgcgcgcggatgctagtacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctattctgaccttgccatcacgactgtgctggtcattaaacgcgtattcaggctgaccctgcgcgctgcgcagggctttattgattccatttttacactgatgaatgttccgttgcgctgcccggattacagatcctctagataatgaattctataggtacctttatggctagctcagtcctaggtacaatgctagcactagagtcacacaggaaaccactagtatgtggagccacccgcagttcgaaaaaggatcaagcaaaggagaagaacttttcactggagttgtcccaattcttgttgaattagatggtgatgttaatgggcacaaattttctgtccgtggagagggtgaaggtgatgctacaaacggaaaacttacccttaaatttatttgcactactggaaaactacctgttccatggccaacacttgtcactactctcacttatggtgttcaatgcttttcccgttatccggatcatatgaaacggcatgactttttcaagagtgccatgcccgaaggttatgtacaggaacgcactatatctttcaaagatgacgggacttacaagacgcgtgctgaagtcaagtttgaaggtgatacccttgttaatcgtatcgagttaaaaggtattgattttaaagaagatggaaacattctcggacacaaacttgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtctacacaatctgtcctttcgaaagatcccaacgaaaagcgtgaccacatggtccttcttgagtttgtaactgctgctgggattacacatggcatggatgagctctacaaataaggatcctgaagatccaacgctcggttgccgccgggcgttttttattctgcaggagcagaagagcatacatctggaagctttctagaatcgggggcccccggggggattctagaacaatgga

SEQ ID NO: 25 - DNA, 8425 bp, synthetic construct
  misc_feature 1..8425: note = plasmid pHM41
  source 1..8425: mol_type = other DNA
agtcctcgtctcgccctcgaattagcccgcttaatgagcgggctttttttgaattaattctcgcgagctggcacgacaggtttcccgactggaaagcgggcagtgagcgcaacgcaattaatgtaagttagcgcgaattgcaagctggccgacgcgctgggctacgtcttgctggcgttcgggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgctggcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccaccatggcatcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgtcggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatcaccgaagcgcttgaccagcccggccatctcgctgcggtactcgtccagcgccgtgcgccggtggcggctaagctgccgctcggacagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtggtggcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgctgggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggcctgcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgaagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggtgcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccctcgcgagaattaattcagataaaaaaaatccttagctttcgctaaggatgatttctagcgatgaccctgctgattggttcgctgaccgtagactggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgacatcgcgcgcgcgcgcgcggatgctagtacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctattctgaccttgccatcacgactgtgctggtcattaaacgcgtattcaggctgaccctgcgcgctgcgcagggctttattgattccatttttacactgatgaatgttccgttgcgctgcccggattacagatcctctagataatgaattctataggtaccttgacagctagctcagtcctaggtataatgctagcactagagacggtaaataacaaaagaggggagggacactagtatgtggagccacccgcagttcgaaaaaggatcaagcaaaggagaagaacttttcactggagttgtcccaattcttgttgaattagatggtgatgttaatgggcacaaattttctgtccgtggagagggtgaaggtgatgctacaaacggaaaacttacccttaaatttatttgcactactggaaaactacctgttccatggccaacacttgtcactactctcacttatggtgttcaatgcttttcccgttatccggatcatatgaaacggcatgactttttcaagagtgccatgcccgaaggttatgtacaggaacgcactatatctttcaaagatgacgggacttacaagacgcgtgctgaagtcaagtttgaaggtgatacccttgttaatcgtatcgagttaaaaggtattgattttaaagaagatggaaacattctcggacacaaacttgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtctacacaatctgtcctttcgaaagatcccaacgaaaagcgtgaccacatggtccttcttgagtttgtaactgctgctgggattacacatggcatggatgagctctacaaataaggatcctgaagatccaacgctcggttgccgccgggcgttttttattctgcaggagcagaagagcatacatctggaagctttctagaatcgggggcccccggggggattctagaacaatgga

SEQ ID NO: 26 - DNA, 7632 bp, synthetic construct
  misc_feature 1..7632: note = plasmid pHM42
  source 1..7632: mol_type = other DNA
agtcctcgtctcgccctcgaattagcccgcttaatgagcgggctttttttgaattaattctcgcgagctggcacgacaggtttcccgactggaaagcgggcagtgagcgcaacgcaattaatgtaagttagcgcgaattgcaagctggccgacgcgctgggctacgtcttgctggcgttcgggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgctggcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccaccatggcatcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgtcggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatcaccgaagcgcttgaccagcccggccatctcgctgcggtactcgtccagcgccgtgcgccggtggcggctaagctgccgctcggacagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtggtggcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgctgggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggcctgcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgaagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggtgcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccctcgcgagaattaattcagataaaaaaaatccttagctttcgctaaggatgatttctagcgatgaccctgctgattggttcgctgaccgtagactggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgacatcgcgcgcgcgcgcgcggatgctagtacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctattctgaccttgccatcacgactgtgctggtcattaaacgcgtattcaggctgaccctgcgcgctgcgcagggctttattgattccatttttacactgatgaatgttccgttgcgctgcccggattacagatcctctagataatgaattctataggtaccataaataaggttttcccctgatgctgccacgcgtgactagtatgtggagccacccgcagttcgaaaaaggatcaagcaaaggagaagaacttttcactggatccaagctttctagaatcgggggcccccggggggattctagaacaatgga

SEQ ID NO: 27 - DNA, 8359 bp, synthetic construct
  misc_feature 1..8359: note = plasmid pHM64
  source 1..8359: mol_type = other DNA
agtcctcgtctcgccctcgaattagcccgcttaatgagcgggctttttttgaattaattctcgcgagctggcacgacaggtttcccgactggaaagcgggcagtgagcgcaacgcaattaatgtaagttagcgcgaattgcaagctggccgacgcgctgggctacgtcttgctggcgttcgggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgctggcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccaccatggcatcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgtcggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatcaccgaagcgcttgaccagcccggccatctcgctgcggtactcgtccagcgccgtgcgccggtggcggctaagctgccgctcggacagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtggtggcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgctgggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggcctgcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgaagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggtgcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccctcgcgagaattaattcagataaaaaaaatccttagctttcgctaaggatgatttctagcgatgaccctgctgattggttcgctgaccgtagactggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgacatcgcgcgcgcgcgcgcggatgctagtacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctattctgaccttgccatcacgactgtgctggtcattaaacgcgtattcaggctgaccctgcgcgctgcgcagggctttattgattccatttttacactgatgaatgttccgttgcgctgcccggattacagatcctctagataatgaattctataggtaccttgacggctagctcagtcctaggtatagtgctagcactagagaaagaggggaaatactagtatgcaccatcaccatcaccatgaaaacctttacttccagagcatggcgactgggtcccgcacttcattgttgctggcatttggacttctttgtctgccttggttgcaggaggggagtgcttttcccactataccattatctcgtctgttcgacaacgcgatgttacgggctcacagacttcaccaattagcttttgatacataccaagagtttgaggaagcctatataccaaaggaacaaaagtattcatttcttcaaaatccgcaaaccagtctttgcttcagcgagagtataccaacaccgtccaaccgtgaggaaacacaacagaagagcaacttagaacttcttcgtatttctcttttgttaatacagtcatggttggaacccgtgcagttcctgagatctgtttttgccaattcactggtctacggtgcatccgatagtaatgtgtacgatttattgaaggacttggaagagggcatccagacgttgatgggtcggcttgaagacggttcgcccagaactggccaaatatttaagcaaacatattccaagttcgacacaaattctcataatgacgatgcactgttgaagaactatggtcttttatactgtttccgcaaggatatggataaagtggaaacatttctgcgtattgtccagtgtcgctcggttgaaggttcctgcggattttagggatcctgaagatccaacgctcggttgccgccgggcgttttttattctgcaggagcagaagagcatacatctggaagctttctagaatcgggggcccccggggggattctagaacaatgga

SEQ ID NO: 28 - DNA, 80 bp, synthetic construct
  misc_feature 1..80: note = Primer DNA_174
  source 1..80: mol_type = other DNA
ataatgaattctataggtaccataaataaggttttcccctgatgctgccacgcgtgactagtatgtggagccacccgcag

SEQ ID NO: 29 - DNA, 39 bp, synthetic construct
  misc_feature 1..39: note = Primer DNA_053
  source 1..39: mol_type = other DNA
tttggtctcaagcttggatccagtgaaaagttcttctcc

SEQ ID NO: 30 - DNA, 71 bp, synthetic construct
  misc_feature 1..71: note = Primer Primer DNA_044
  source 1..71: mol_type = other DNA
tgtccctcccctcttttgttatttaccgtctctagtgctagcattatacctaggactgagctagctgtcaa

SEQ ID NO: 31 - DNA, 20 bp, synthetic construct
  source 1..20: mol_type = other DNA
atacttgattcccgcacacc

SEQ ID NO: 32 - DNA, 20 bp, synthetic construct
  source 1..20: mol_type = other DNA
gtcagttcaacgagcaaagc